(19) United States
(12) Patent Application Publication
(10) Pub. No.: US 2011/0200619 A1
(43) Pub. Date: Aug. 18, 2011

(54) PHARMACEUTICAL COMPOUNDS AS CYTOTOXIC AGENTS AND USES THEREOF

(75) Inventors: Mark B. Anderson, Orlinda, CA (US); In Chul Kim, Salt Lake City, UT (US)

(73) Assignee: Myrexis, Inc., Salt Lake City, UT (US)

(21) Appl. No.: 13/004,311

(22) Filed: Jan. 11, 2011

Related U.S. Application Data
(63) Continuation of application No. PCT/US2009/050036, filed on Jul. 9, 2009.
(60) Provisional application No. 61/079,887, filed on Jul. 11, 2008.

(51) Int. Cl. A61K 39/395; A61K 31/517; A61K 38/18; A61K 38/19; C07D 239/94; A61P 35/00

(52) U.S. Cl. 424/174.1; 514/266.4; 424/130.1; 514/7.6; 514/1.1; 544/287

(57) ABSTRACT

Disclosed are compounds effective as cytotoxic agents. The compounds of this invention are useful in the treatment of a variety of clinical conditions in which uncontrolled growth and spread of abnormal cells occurs.

### CROSS-REFERENCE TO RELATED APPLICATIONS

This application is a continuation of International Application No. PCT/US2009/050036, filed Jul. 9, 2009, which claims the benefit of U.S. Provisional Application No. 61/079,887, filed Jul. 11, 2008, the contents of both of which are hereby incorporated by reference in their entirety.

### FIELD OF THE INVENTION

This invention is in the field of medicinal chemistry. In particular, the invention relates to compounds that are cytotoxic agents. The invention also relates to the use of these compounds as therapeutically effective anti-cancer agents.

### BACKGROUND OF THE INVENTION

Cancer is a common cause of death in the world; about 10 million new cases occur each year, and cancer is responsible for 12% of deaths worldwide, making cancer the third leading cause of death. World Health Organization, National Cancer Control Programmes Policies and Managerial Guidelines (2d ed. 2002).

Despite advances in the field of cancer treatment, the leading therapies to date include surgery, radiation, and chemotherapy. Chemotherapeutic approaches are said to fight cancers that are metastasized or that are particularly aggressive. Most of the cancer chemotherapy agents currently in clinical use are cytotoxins. Cytotoxic agents work by damaging or killing cells that exhibit rapid growth. Ideal cytotoxic agents would have specificity for cancer and tumor cells, while not affecting normal cells. Unfortunately, none have been found and instead agents that target especially rapidly dividing cells (both tumor and normal) have been used.

Accordingly, discovery of new and effective treatments for cancer is a high priority for health care researchers. Materials that are cytotoxic to cancer cells while exerting only mild effects on normal cells are highly desirable. For this reason, there remains a definite need in the art for new effective chemotherapeutic agents.

### BRIEF SUMMARY OF THE INVENTION

The present invention is related to the discovery that (2-aminomethylquinazolin-4-yl)-(4-methoxyphenyl)methylamine is a cytotoxic agent. Thus, (2-aminomethylquinazolin-4-yl)-(4-methoxyphenyl)methylamine is useful in treating or delaying the onset of diseases and disorders that are responsive to cytotoxic agents.

Accordingly, one aspect of the present invention is directed to the use of the compound of the present invention in treating or ameliorating neoplasm and cancer, by administering the compound to cells in vitro or in vivo in warm-blooded animals, particularly mammals.

(2-aminomethylquinazolin-4-yl)-(4-methoxyphenyl)methylamine is a novel compound. Therefore, another aspect of the present invention is to provide a novel compound.

Yet another aspect of the present invention is to provide a pharmaceutical composition useful for treating disorders responsive to cytotoxic agents, containing an effective amount of the compound of the present invention, preferably in admixture with one or more pharmaceutically acceptable carriers or diluents.

In yet another aspect of the present invention, methods are provided for the preparation of the novel compound of the present invention.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

It has been discovered that the compound of the present invention is a potent and highly efficacious cytotoxic agent. Therefore, the compound is useful for treating diseases and disorders responsive to cytotoxic agents.

The above various methods of the present invention can be practiced by or comprise treating cells in vitro or a warm-blooded animal, particularly mammal, more particularly a human, with an effective amount of a compound according to the present invention. As used herein, the phrase “treating . . . with . . . a compound” means either administering the compound to cells or an animal, or administering to cells or an animal the compound or another agent to cause the presence or formation of the compound inside the cells or the animal. Preferably, the methods of the present invention comprise administering to cells in vitro or to a warm-blooded animal, particularly mammal, more particularly a human, a pharmaceutical composition comprising an effective amount of a compound according to the present invention.

Specifically, the methods of the present invention comprise treating cells in vitro or a warm-blooded animal, particularly mammal, more particularly a human, with an effective amount of (2-aminomethylquinazolin-4-yl)-(4-methoxyphenyl)methylamine.

The invention includes all stereoisomers and both the racemic mixtures of such stereoisomers as well as the individual enantiomers that may be separated according to methods that are well known to those of ordinary skill in the art.

Examples of pharmaceutically acceptable addition salts include inorganic and organic acid addition salts, and inorganic and organic base addition salts.

In the compounds of the invention, reference to any bound hydrogen atom can also encompass a deuterium atom bound at the same position. Substitution of hydrogen atoms with deuterium atoms is conventional in the art. See, e.g., U.S. Pat. Nos. 5,149,820 & 7,317,039. Such deuteration sometimes results in a compound that is functionally indistinct from its hydrogenated counterpart, but occasionally results in a compound having beneficial changes in the properties relative to the non-deuterated form. For example, in certain instances, replacement of specific bound hydrogen atoms with deuterium atoms dramatically slows the catabolism of the deuterated compound, relative to the non-deuterated compound, such that the deuterated compound exhibit a significantly longer half-life in the bodies of individuals administered such compounds. This is particulary the case when the catabolism of the hydrogenated compound is mediated by cytochrome P450 systems. Kushner et al., Can. J. Physiol. Pharmacol. (1999) 77:79-88.

The compounds of this invention may be prepared using methods known to those skilled in the art, or the novel methods of this invention. In one embodiment, the compound (2-aminomethylquinazolin-4-yl)-(4-methoxyphenyl)methylamine, or a pharmaceutically acceptable salt thereof, is prepared by a method comprising reacting 4-chloro-2-chloromethylquinazoline, or a salt thereof, with (4-methoxyphenyl)methylamine, or a salt thereof, under suitable conditions and with suitable reagents to form a first intermediate, (2-chloromethylquinazolin-4-yl)(4-methoxyphenyl)methylamine, or a salt thereof. The method further comprises reacting said first intermediate with a phthalimide salt under suitable conditions and with suitable reagents to form a second intermediate, 2-{4-[(4-methoxyphenyl)methylamino]quinazolin-2-ylmethyl}isoindole-1,3-dione, or a salt thereof. The method further comprises reacting said second intermediate with an amine base under suitable conditions and with suitable reagents to form (2-aminomethylquinazolin-4-yl)-(4-methoxyphenyl)methylamine, or a pharmaceutically acceptable salt thereof.

In one embodiment, the compound (2-aminomethylquinazolin-4-yl)-(4-methoxyphenyl)methylamine, or a pharmaceutically acceptable salt thereof, is prepared by a method comprising reacting 2,4-dichloro-quinazoline, or a salt thereof, and 4-methoxy-N-methylaniline, or a salt thereof, under suitable conditions and with suitable reagents to form a first intermediate, (2-chloroquinazolin-4-yl)(4-methoxyphenyl)methylamine, or a salt thereof. The method further comprises reacting said first intermediate with a cyanide salt under suitable conditions and with suitable reagents to form a second intermediate, 4-[(4-methoxyphenyl)-methylamino]-quinazoline-2-carbonitrile, or a salt thereof. The method further comprises reducing the carbonitrile moiety of said second intermediate with under suitable conditions and with suitable reagents to form (2-aminomethylquinazolin-4-yl)-(4-methoxyphenyl)methylamine, or a pharmaceutically acceptable salt thereof.

In other embodiments, (2-aminomethylquinazolin-4-yl)-(4-methoxyphenyl)methylamine can be prepared as illustrated by the exemplary reactions in Schemes 1-2, below.

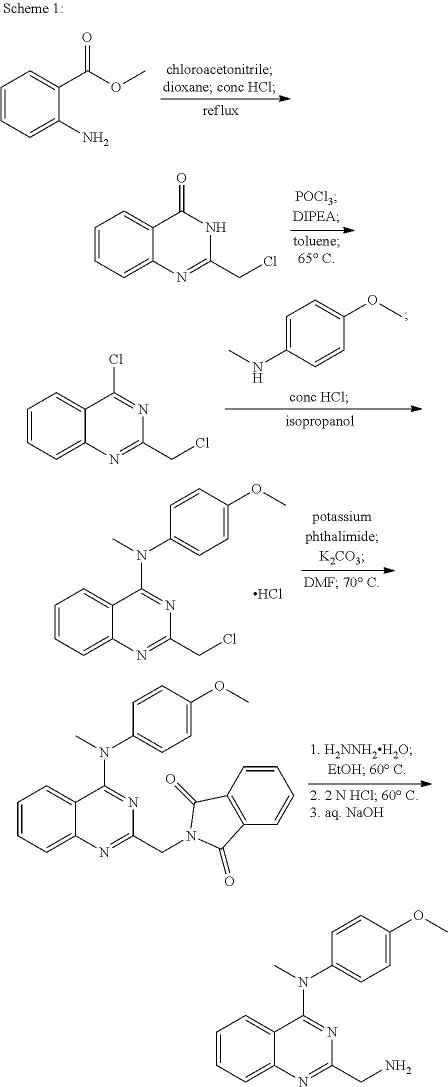

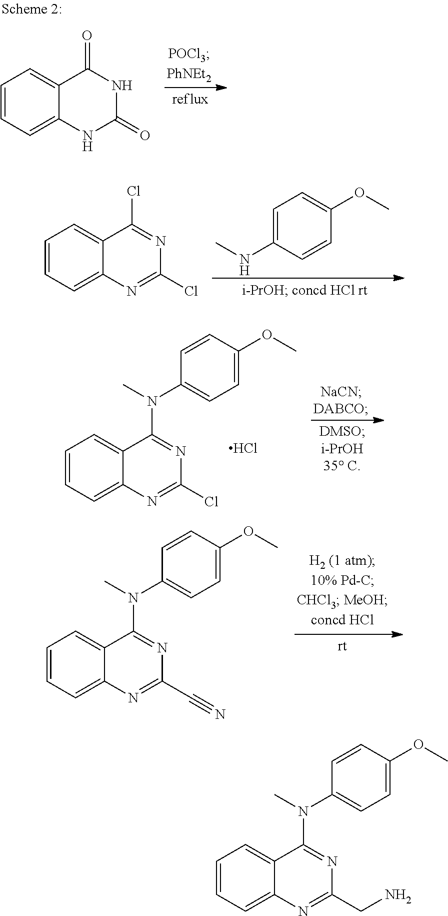

General: ¹H NMR were recorded at 400 MHz. MPLC were run on silica cartridges supplied by Teledyne Isco. Preparative TLC plates are Silica Gel GF 1000 μm 20×20 cm (Analtech 02013). Preparative RPLC were typically run on C₁₈ columns using a gradient of (0.01% TFA in acetonitrile) against (0.01% TFA in water).

### Scheme 1:

2-Chloromethyl-3H-quinazolin-4-one: A solution of methyl anthranilate (10.0 mL, 77.3 mmol) and chloroacetonitrile (5.5 mL, 87.1 mmol) in dioxane (150 mL) was treated with HCl (12 N, 10 mL, 120 mmol) and the resulting suspension refluxed overnight. The suspension was cooled to room temperature (rt) and the solid collected via vacuum filtration and washed with hexanes. The solid thus obtained was suspended in H₂O and neutralized with NaHCO₃. The solid was collected via vacuum filtration and dried under vacuum to yield 8.864 g (59%) of the title compound as a white solid. ¹H NMR (DMSO-d₆) δ 12.6 (s (br), 1H), 8.13 (ddd, 1H), 7.85 (ddd, 1H), 7.69 (ddd, 1H), 7.56 (ddd, 1H), 4.56 (s, 2H); LC-MS (ESI⁺; 195 ([M+H]⁺)).

4-Chloro-2-chloromethylquinazoline: A suspension of 2-chloromethyl-3H-quinazolin-4-one (12.27 g) in toluene (200 mL) was treated with Hünig's base (19 mL, 109 mmol) and POCl₃ (8.8 mL, 96.1 mmol) and heated to 65° C. overnight. The reaction was cooled to rt and the layers separated. The bottom layer was extracted with toluene. The top layers were combined and washed with cold H₂O and sat. NaHCO₃, dried (MgSO₄), filtered and concentrated. Purification by gradient MPLC (SiO₂, 120 g column, EtOAc/hexanes, 0-100%) provided 9.72 g (69%) of the title compound as a white solid. ¹H NMR (DMSO-d₆) δ 8.33 (ddd, 1H), 8.05-8.22 (m, 2H), 7.93 (ddd, 1H), 4.97 (s, 2H); LC-MS (ESI⁺; 213 ([M+H]⁺)).

(2-Chloromethylquinazolin-4-yl)(4-methoxyphenyl)methylamine hydrochloride: A suspension of 4-chloro-2-chloromethylquinazoline (7.383 g, 35.0 mmol) and (4-methoxyphenyl)methylamine (4.837 g, 35.3 mmol) in i-PrOH (50 mL) was treated with HCl (12 M, 1.5 mL, 18 mmol) and stirred at rt for 2 h. The resulting solid was collected by vacuum filtration, yielding 10.367 g (85%) of the title compound. ¹H NMR (DMSO-d₆) δ 7.80-7.94 (m, 2H), 7.40-7.80 (m, 2H), 7.26-7.34 (m, 1H), 7.07-7.15 (m, 2H), 6.83 (br d, 1H), 4.94 (s, 2H), 3.83 (s, 3H), 3.72 (s, 3H); LC-MS (ESI⁺; 314 ([M+H]⁺)).

2-{4-[(4-Methoxyphenyl)methylamino]quinazolin-2-ylmethyl}isoindole-1,3-dione: A suspension of (2-chloromethylquinazolin-4-yl)(4-methoxyphenyl)methylamine hydrochloride (10.367 g, 16.2 mmol) and K₂CO₃ (2.25 g, 16.3 mmol) in DMF (50 mL) was heated to 70° C. for 1 h. The reaction was cooled to rt, potassium phthalimide (6.004 g, 32.5 mmol) was added and the reaction heated to 70° C. for 2 h. The reaction was cooled to rt, diluted with EtOAc, washed with H₂O and 5% NaOH, dried (MgSO₄), filtered and concentrated. The residue was purified by gradient MPLC (SiO₂, EtOAc/hexanes 0-100%) to yield 8.56 g (68%) of the title compound. ¹H NMR (DMSO-d₆) δ 7.95-8.02 (m, 2H), 7.87-7.94 (m, 2H), 7.55-7.60 (m, 2H), 7.18-7.22 (m, 2H), 7.02-7.12 (m, 1H), 6.94-7.01 (m, 2H), 6.88 (dt, 1H), 4.95 (s, 2H), 3.77 (s, 3H), 3.26 (s, 3H). HRMS (ES) calcd for C₂₅H₂₁N₄O₃ (M+H) 425.1608, found 425.1604.

2-(Aminomethyl)-N-(4-methoxyphenyl)-N-methylquinazolin-4-amine: A solution of 2-{4-[(4-methoxyphenyl)methylamino]quinazolin-2-ylmethyl}isoindole-1,3-dione (8.561 g, 20.2 mmol) in EtOH (100 mL) was treated with hydrazine mono-hydrate (3.0 mL, 61.8 mmol) and heated to 60° C. for 2 h. The reaction was cooled to rt, HCl (2 N, 50 mL) added and the reaction heated to 60° C. for 30 min. After cooling to rt the solid was filtered off. The filtrate was concentrated, basified with 5% NaOH and extracted with CH₂Cl₂. The organic layers were combined, dried (MgSO₄), filtered and concentrated. The residue was purified by gradient reverse phase MPLC (MeCN/H₂O with 0.1% TFA) and the free base re-extracted as above to yield 3.10 g (52%) of the title compound. ¹H NMR (CDCl₃) δ 7.76 (d, 1H), 7.54 (ddd, 1H), 7.08-7.16 (m, 2H), 6.95-7.05 (m, 2H), 6.86-6.94 (m, 2H), 4.07 (s, 2H), 3.84 (s, 3H), 3.60 (s, 3 H), 2.00 (s (br), 2H). ¹³C NMR (CDCl₃) δ 165.9, 162.0, 158.2, 152.2, 141.6, 132.0, 128.2, 127.6, 126.5, 124.4, 115.5, 115.4, 55.7, 49.1, 43.0. HRMS (ES) calcd for C₁₇H₁₈N₄O (M+H) 295.1553, found 295.1506.

### Scheme 2:

2,4-Dichloro-quinazoline: A suspension of 1H-quinazoline-2,4-dione (10 g, 62 mmol), POCl₃ (50 mL, 546 mmol) and N,N-dimethylaniline (1 mL, 7.9 mmol) was heated to reflux for 18 h. The reaction mixture was cooled to room temperature and poured slowly onto ice and extracted with CH₂Cl₂. The combined extracts were filtered through Na₂SO₄ and concentrated to give 4.2 g (34%) of 2,4-dichloro-quinazoline as a white solid.

(2-Chloro-quinazolin-4-yl)-(4-methoxy-phenyl)-methyl-amine hydrochloride: A solution of 2,4-dichloro-quinazoline (1 g, 5 mmol) and 4-methoxy-N-methylaniline (0.823 g, 6 mmol) in i-PrOH (17 mL) with HCl (12 M, 10 drops) was stirred at room temperature overnight. The reaction was filtered, washed with i-PrOH and dried under vacuum to provide 1 g (66%) of the title compound as a white solid. ¹H NMR (CDCl₃) δ 8.65 (d, 1H), 7.7 (t, 1H), 7.23 (d, 2H), 7.18 (t, 1H), 7.08 (d, 2H), 6.75 (d, 1H), 3.9 (s, 3H) 3.8 (s, 3H).

4-[(4-Methoxyphenyl)(methyl)amino]quinazoline-2-carbonitrile: To a solution of (2-chloroquinazolin-4-yl)-(4-methoxyphenyl)-methylamine hydrochloride (56 mg, 0.17 mmol) in 1 mL of DMSO and 0.5 mL of 2-propanol were added sodium cyanide (16 mg, 0.34 mmol) and 1,4-diazabicyclo[2.2.2]octane (9 mg, 0.084 mmol). The mixture was heated at 35° C. for 1 day with stirring. The solution was diluted with 5 mL of water and 10 mL of ethyl acetate. The organic layer was washed with water, then dried and concentrated. The resulting crude material was purified by silica gel column chromatography to give the title compound (34 mg, 70%). ¹H NMR (CDCl₃) δ 7.85 (d, 1H, J=8.4 Hz), 7.64 (t, 1H, J=6.8 Hz), 7.14 (m, 3H), 6.96 (m, 3H), 3.86 (s, 3H), 3.61 (s, 3H); LC-MS (ESI⁺; 291 ([M+H]⁺)).

(2-aminomethylquinazolin-4-yl)-(4-methoxyphenyl)methylamine: A mixture of 4-[(4-methoxyphenyl)-methylamino]-quinazoline-2-carbonitrile (22 mg, 0.076 mmol), 10% palladium on carbon (11 mg), concd HCl (0.05 mL) in 1 mL of chloroform and 5 mL of methanol was stirred under hydrogen (1 atm) overnight. The solution was passed through a pad of Celite and then concentrated. The resulting crude material was purified by MPLC (SiO₂/gradient of (1:1:8 Et₃N:MeOH:EtOAc) in EtOAc) to give the title compound (20 mg, 90%). ¹H NMR (CDCl₃) δ 7.74 (d, 1H, J=8.4 Hz), 7.53 (t, 1H, J=6.8 Hz), 7.11 (d, 2H, J=8.8 Hz), 7.00 (m, 2H), 6.90 (d, 2H, J=8.8 Hz), 4.06 (s, 2H), 3.83 (s, 3H), 3.59 (s, 3H); LC-MS (ESI⁺; 295 ([M+H]⁺)).

An important aspect of the present invention is the discovery that (2-aminomethylquinazolin-4-yl)-(4-methoxyphenyl)methylamine is a cytotoxic agent. Therefore, (2-aminomethylquinazolin-4-yl)-(4-methoxyphenyl)methylamine is useful in treating diseases that are responsive to cytotoxic agents. For example, (2-aminomethylquinazolin-4-yl)-(4-methoxyphenyl)methylamine is useful in the treatment of a variety of clinical conditions in which there is uncontrolled cell growth and spread of abnormal cells, such as in the case of neoplasia or cancer. Examples of such cancers include, but are not limited to, such specific diseases as Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma. Importantly, (2-aminomethylquinazolin-4-yl)-(4-methoxyphenyl)methylamine can be used in the treatment of primary cancers, or in the treatment of metastatic cancers, such as metastatic brain cancer.

Thus, the present invention includes therapeutic methods for the treatment of a variety of cancer types, comprising administering to an animal in need of such treatment an effective amount of (2-aminomethylquinazolin-4-yl)-(4-methoxyphenyl)methylamine, or a pharmaceutically acceptable salt thereof, wherein said therapeutic method is useful to treat the cancer present. Such cancers being a group of diseases characterized by the uncontrolled growth and spread of abnormal cells.

In practicing the therapeutic methods of the invention, effective amounts of compositions containing therapeutically effective concentrations of the compounds formulated for oral, intravenous, local and topical application, for the treatment of neoplastic diseases and other diseases, including a variety of cancers, are administered to an individual exhibiting the symptoms of one or more of these neoplastic diseases. The effective amounts are effective in reducing, ameliorating, or eliminating one or more symptoms of the neoplastic disease. An effective amount of a compound for treating a particular disease is an amount that is sufficient to ameliorate, or in some manner reduce, the symptoms associated with that disease. Such an effective amount may be administered as a single dose or may be administered according to a dosage regimen, chosen for enhanced effectiveness. The effective amount of (2-aminomethylquinazolin-4-yl)-(4-methoxyphenyl)methylamine may cure the disease but, typically, is administered in order to ameliorate the symptoms of the disease. Often, repeated administration is required to achieve the desired amelioration of symptoms or cure of the disease.

Another aspect of the present invention is to provide a pharmaceutical composition, containing an effective amount of (2-aminomethylquinazolin-4-yl)-(4-methoxyphenyl)methylamine, or a pharmaceutically acceptable salt thereof, in admixture with one or more pharmaceutically acceptable carriers or diluents.

In one embodiment, a pharmaceutical composition comprising (2-aminomethylquinazolin-4-yl)-(4-methoxyphenyl)methylamine, or a pharmaceutically acceptable salt thereof, in combination with a pharmaceutically acceptable vehicle is provided.

Another embodiment of the present invention is directed to a composition effective to inhibit neoplasia comprising (2-aminomethylquinazolin-4-yl)-(4-methoxyphenyl)methylamine, or a pharmaceutically acceptable salt thereof, which functions as a cytotoxic agent, in combination with at least one known cancer chemotherapeutic agent, or a pharmaceutically acceptable salt of said agent. Examples of known cancer chemotherapeutic agents which may be used for combination therapy include, but not are limited to alkylating agents, antimitotic agents, topoisomerase I inhibitors, topoisomerase II inhibitors, RNA/DNA antimetabolites, DNA antimetabolites, EGFR inhibitors, proteosome inhibitors, and antibodies.

In practicing the methods of the present invention, the compound of the invention may be administered together with at least one known chemotherapeutic agent as part of a unitary pharmaceutical composition. Alternatively, the compound of the invention may be administered apart from at least one known cancer chemotherapeutic agent. In one embodiment, the compound of the invention and at least one known cancer chemotherapeutic agent are administered substantially simultaneously, i.e. the compounds are administered at the same time or one after the other, so long as the compounds reach therapeutic levels in the blood at the same time. In another embodiment, the compound of the invention and at least one known cancer chemotherapeutic agent are administered according to their individual dosing schedules, so long as the compounds ultimately reach therapeutic levels in the blood.

Another embodiment of the present invention is directed to a composition effective to inhibit neoplasia comprising a bioconjugate of a compound described herein, which functions as a cytotoxic agent, in bioconjugation with at least one known therapeutically useful antibody, growth factor, cytokine, or any molecule that binds to the cell surface. The antibodies and other molecules can assist in the delivery of the compound described herein to its target(s) and can improve the efficacy of the compound as an anticancer agent. The bioconjugates can also enhance the anticancer effect of therapeutically effective antibodies.

Similarly, another embodiment of the present invention is directed to a composition effective to inhibit neoplasia comprising a compound described herein, or a pharmaceutically acceptable salt of a compound described herein, which functions as a cytotoxic agent, in combination with radiation therapy. In this embodiment, the compound of the invention may be administered at the same time as the radiation therapy is administered, or at a different time from the administration of radiation therapy.

Yet another embodiment of the present invention is directed to a composition effective for post-surgical treatment of cancer, comprising a compound described herein, or a pharmaceutically acceptable salt of a compound described herein, which functions as a cytotoxic agent. The invention also relates to a method of treating cancer by surgically removing the cancer and then treating the animal with one of the pharmaceutical compositions described herein.

Stent implantation has become the new standard angioplasty procedure. However, in-stent restenosis remains the major limitation of coronary stenting. New approaches have been developed to target pharmacological modulation of local vascular biology by local administration of drugs. This allows for drug applications at the precise site and time of vessel injury. Numerous pharmacological agents with antiproliferative properties are currently under clinical investigation, including actinomycin D, rapamycin or paclitaxel coated stents (Regar E., et al., Br. Med. Bull. 59:227-248 (2001)). Therefore, apoptosis inducers, which are antiproliferative, are useful as therapeutics for the prevention or reduction of in-stent restenosis.

Multidrug resistance (MDR) is the major cause of cancer chemotherapy failure. Drug resistance is typically caused by ATP-dependent efflux of drug from cells by ATP-binding cassette (ABC) transporters. In particular, the ABC transporters ABCB1 (MDR-1, P glycoprotein); ABCC1 (MRP1); and ABCG2 (BCRP, MXR) are typically over-expressed in drug resistant tumors and thus are implicated in drug resistance. In comparison to most standard anti-cancer drugs, which are not effective in killing drug resistant cancer cells, the compound of the present invention is effective in killing drug resistant cancer cells. Therefore, the compound of this invention is useful for the treatment of drug resistant cancer.

Hence, another important aspect of the present invention is the surprising discovery that the compound of the present invention is a potent and highly efficacious cytotoxic agent even in drug resistant cancer cells, which enables the compound to inhibit the growth and proliferation of drug resistant cancer cells, and to cause cell death in the drug resistant cancer cells. Specifically, the compound of the present invention is not a substrate for the multidrug resistance transporters such as Pgp-1 (MDR-1), MRP-1 and BCRP. This is particularly surprising in view of the fact that many commercially available chemotherapeutics are substrates for such MDR transporters.

Thus, another aspect of the present invention is the application of the methods and the compound of the present invention as described above to treat or ameliorate tumors that have acquired resistance to other anticancer drugs. In one embodiment of this aspect of the invention, the compound of the present invention is administered to a cancer patient who has been treated with another anti-cancer drug. In another embodiment, the compound of the present invention is administered to a patient who has been treated with and is not responsive to another anti-cancer drug or developed resistance to such other anti-cancer compound. In another embodiment, the compound of the present invention is administered to a patient who has been treated with another anti-cancer drug and is refractory to said other anti-cancer drug. The compound of the present invention can be used in treating cancer in a patient who is not responsive or is resistant to any other anti-cancer agent. Examples of such other anti-cancer agent may include alkylating agents, antimitotic agents, topoisomerase I inhibitors, topoisomerase II inhibitors, RNA/DNA antimetabolites, EGFR inhibitors, angiogenesis inhibitors, tubulin inhibitors, proteosome inhibitors, etc.

Pharmaceutical compositions within the scope of this invention include all compositions wherein the compound of the present invention is contained in an amount that is effective to achieve its intended purpose. While individual needs vary, determination of optimal ranges of effective amounts of each component is within the skill of the art. Typically, the compound of the invention may be administered to animals, e.g., mammals, orally at a dose of 0.0025 to 50 mg/kg of body weight, per day, or an equivalent amount of the pharmaceutically acceptable salt of the compound of the invention may be administered, to a mammal in need of treatment. Preferably, the compound of the invention, or an equivalent amount of a pharmaceutically acceptable salt thereof, is orally administered at a dose of approximately 0.01 to approximately 10 mg/kg of body weight. For intramuscular injection, the dose is generally approximately one-half of the oral dose. For example, a suitable intramuscular dose would be approximately 0.0025 to approximately 25 mg/kg of body weight, and most preferably, from approximately 0.01 to approximately 5 mg/kg of body weight. If a known cancer chemotherapeutic agent is also administered, it is administered in an amount that is effective to achieve its intended purpose. The amounts of such known cancer chemotherapeutic agents effective for cancer treatment are well known to those skilled in the art.

The unit oral dose may comprise from approximately 0.01 to approximately 50 mg, preferably approximately 0.1 to approximately 10 mg of the compound of the invention, or an equivalent amount of a pharmaceutically acceptable salt thereof. The unit dose may be administered one or more times daily, as one or more tablets, each containing from approximately 0.1 to approximately 10 mg, conveniently approximately 0.25 to 50 mg of the compound or its salts or solvates.

In a topical formulation, the compound of the invention may be present at a concentration of approximately 0.01 to 100 mg per gram of carrier.

In addition to administering the compound as a raw chemical, the compound of the invention may be administered as part of a pharmaceutical preparation containing suitable pharmaceutically acceptable carriers comprising excipients and auxiliaries, which facilitate processing of the compound into preparations that may be used pharmaceutically. Preferably, the preparations, particularly those preparations which may be administered orally and that may be used for the preferred type of administration, such as tablets, dragees, and capsules, and also preparations that may be administered rectally, such as suppositories, as well as suitable solutions for administration orally, or by injection, contain from approximately 0.01 to 99 percent, preferably from approximately 0.25 to 75 percent of active compound, together with the excipient(s).

Also included within the scope of the present invention are the non-toxic pharmaceutically acceptable salts of the compound of the present invention. Acid addition salts are formed by mixing a solution of the compound of the present invention with a solution of a pharmaceutically acceptable non-toxic acid. Basic salts are formed by mixing a solution of the compound of the present invention with a solution of a pharmaceutically acceptable non-toxic base.

The pharmaceutical compositions of the invention may be administered to any animal, which may experience the beneficial effects of the compound of the invention. Foremost among such animals are mammals, e.g., humans and veterinary animals, although the invention is not intended to be so limited.

While the pharmaceutical compositions of the present invention are preferably administered orally, the pharmaceutical compositions of the present invention may be administered by any means and any route that serves to achieve the intended purpose. For example, administration may be by parenteral, subcutaneous, intravenous, intramuscular, intraperitoneal, transdermal, buccal, intrathecal, intracranial, intranasal, rectally, or topical routes. Such alternative routes of administration may be employed exclusively or concurrently with administration by the oral route. The dosage administered orally and via these other routes will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use may be obtained by combining the active compounds with solid excipients, optionally blending and grinding the resulting mixture and processing the mixture of granules, after adding suitable auxiliaries, if desired or necessary, to obtain final blend(s) used to make tablets or dragee cores.

Suitable excipients are, in particular: fillers, cellulose preparations and/or calcium phosphates, as well as binders. If desired, disintegrating agents may be added, such as starches and also carboxymethyl-starch, cross-linked polyvinyl pyrrolidone, agar, or alginic acid or a salt thereof. Auxiliaries are, above all, flow-regulating agents and lubricants. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations are used. Dye stuffs or pigments may be added to the tablets or dragee coatings, e.g., for identification or in order to characterize combinations of active compound doses.

Other pharmaceutical preparations, which may be used orally include push-fit capsules made of gelatin, as well as soft, sealed capsules made of gelatin and a plasticizer. The push-fit capsules may contain the active compounds in the form of: granules, which may be mixed with fillers, binders, and/or lubricants, and, optionally, stabilizers. In soft capsules, the active compounds are preferably dissolved or suspended in suitable liquids, such as fatty oils, or liquid paraffin. In addition, stabilizers may be added.

Possible pharmaceutical preparations, which may be used rectally include, e.g., suppositories, consisting of a combination of one or more of the active compounds with a suppository base. Suitable suppository bases are, e.g., natural or synthetic triglycerides, or paraffin hydrocarbons. In addition, it is also possible to use gelatin rectal capsules, which consist of a combination of the active compounds with a base. Possible base materials include, e.g., liquid triglycerides, polyethylene glycols, or paraffin hydrocarbons.

Suitable formulations for parenteral administration include aqueous solutions of the active compounds in water-soluble form, e.g., water-soluble salts and alkaline solutions. In addition, suspensions of the active compounds as appropriate oily injection suspensions may be administered. Suitable lipophilic solvents or vehicles include fatty oils or synthetic fatty acid esters. Aqueous injection suspensions may contain substances which increase the viscosity of the suspension include. Optionally, the suspension may also contain stabilizers.

In accordance with one aspect of the present invention, the compound of the invention is employed in topical and parenteral formulations and is used for the treatment of skin cancer.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which mixture of the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### Example 1

### Identification of Cytotoxic Agents

A P388 murine leukemia cell line was obtained from NCI, Frederick, Md. P388 cells were cultured in RPMI-1640 supplemented with 10% fetal bovine serum, 2 mM Glutamax, 1 mM sodium pyruvate, 0.1 mM non-essential amino acids and 10 mM HEPES. Cells were grown at 37° C. in a humidified 5% CO₂ atmosphere. Exponentially growing P388 cells were plated at 5,000 cells/well in a 96-well flat-bottomed microtiter plate (Corning, Costar #3595). Twenty-four hours later, test compound was added to cells at final concentrations of 100 nM, 33.3 nM, 11.1 nM, 3.7 nM, 1.23 nM, 0.4 nM and 0.13 nM. Cellular viability was determined 72 hours later by measuring intracellular ATP with ATP-Lite assay system. The effect of compounds on cell viability was calculated by comparing the ATP levels of cells exposed to test compound with those of cells exposed to DMSO. A semi-log plot of relative ATP levels versus compound concentration was used to calculate the compound concentration required to inhibit growth by 50% (IC50). Data was analyzed by Prism software (GraphPad; San Diego, Calif.) by fitting it to a sigmoidal dose response curve.

The P388 IC50 data for (2-aminomethylquinazolin-4-yl)-(4-methoxyphenyl)methylamine was found to be 20 nM. Accordingly, compounds of the invention were identified as cytotoxic agents and are thus useful in treating the various diseases and disorders discussed above.

### Example 2

### Multidrug Resistant Cell Assays

Cytotoxicity of the compound in multidrug resistant cells was determined by administering the compound to cell lines that overexpress the multidrug resistance pump MDR-1 and determining the viability of the cell lines. P388/ADR cell lines are known to overexpress the multidrug resistance pump MDR-1 (also known as P-glycoprotein-1; Pgp-1).

P388/ADR cell lines were obtained from American Type Culture Collection (Manassas, Va.) and maintained in RPMI-1640 media supplemented with 10% FCS, 10 units/ml penicillin and streptomycin, 2 mM Glutamax and 1 mM sodium pyruvate (Invitrogen Corporation, Carlsbad, Calif.). For compound testing, cells were plated in 96 well dishes at a concentration of 1.5×10⁴ cells/well. Cells were allowed to adhere to the plate overnight and then incubated with the compound at final concentrations ranging from 0.13 nM to 10 uM for 72 hours. Cell viability was then assessed using the ATP-lite reagent (Perkin Elmer, Foster City, Calif.). Plates were read on a Wallac Topcount luminescence reader (Perkin Elmer, Foster City, Calif.) and the results graphed in Prism software (Graphpad Software, Inc., San Diego, Calif.). Non-linear regression with variable slope analysis was performed to obtain IC50 concentration values.

The P388/MDR IC50 data for (2-aminomethylquinazolin-4-yl)-(4-methoxyphenyl)methylamine was found to be 18 nM. Accordingly, (2-aminomethylquinazolin-4-yl)-(4-methoxyphenyl)methylamine was identified as a cytotoxic agent in multidrug resistant cells and is thus useful in treating the various diseases and disorders discussed above in drug resistant cancer patients.

### Example 3

Injection Formulation

| Components | Amount | |
|---|---|---|
| Active Compound | 5 | mg |
| PEG-400 | 5 | grams |
| TPGS | 10 | grams |
| Benzyl alcohol | 0.5 | gram |
| Ethanol | 2 | grams |

| | |
|---|---|
| D5W | Add to make 50 mL |

An injection formulation of (2-aminomethylquinazolin-4-yl)-(4-methoxyphenyl)methylamine (the “Active Compound”) can be prepared according to the following method. 5 mg of the Active Compound is dissolved into a mixture of the d-α-tocopheryl polyethylene glycol 1000 succinate (TPGS), PEG-400, ethanol, and benzyl alcohol. D5W is added to make a total volume of 50 mL and the solution is mixed. The resulting solution is filtered through a 0.2 μm disposable filter unit and is stored at 25° C. Solutions of varying strengths and volumes are prepared by altering the ratio of Active Compound in the mixture or changing the total amount of the solution.

### Example 4

Tablet Formulation

| Components | Amount | |
|---|---|---|
| Active Compound | 100.0 | mg |
| Lactose | 100.0 | mg |
| Corn Starch | 50.0 | mg |
| Hydrogenated Vegetable Oil | 10.0 | mg |
| Polyvinylpyrrolidone | 10.0 | mg |
| | 270.0 | mg |

A formulation of tablets of (2-aminomethylquinazolin-4-yl)-(4-methoxyphenyl)methylamine (the “Active Compound”) can be prepared according to the following method. 100 mg of Active Compound) is mixed with 100 mg lactose. A suitable amount of water for drying is added and the mixture is dried. The mixture is then blended with 50 mg of corn starch, 10 mg hydrogenated vegetable oil, and 10 mg polyvinylpyrrolidinone. The resulting granules are compressed into tablets. Tablets of varying strengths are prepared by altering the ratio of Active Compound in the mixture or changing the total weight of the tablet.

### Example 5

Capsule Formulation

| Components | Amount |
|---|---|
| Active Compound | 100.0 mg |
| Microcrystalline Cellulose | 200.0 mg |
| Corn Starch | 100.0 mg |
| Magnesium Stearate | 400.0 mg |
| | 800.0 mg |

A formulation of capsules containing 100.0 mg of (2-aminomethylquinazolin-4-yl)-(4-methoxyphenyl)methylamine (the “Active Compound”) can be prepared according to the following method. 100 mg of Active Compound is mixed with 200 mg of microcrystalline cellulose and 100 mg of corn starch. 400 mg of magnesium stearate is then blended into the mixture and the resulting blend is encapsulated into a gelatin capsule. Doses of varying strengths can be prepared by altering the ratio of the Active Compound to pharmaceutically acceptable carriers or changing the size of the capsule.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. The compound (2-aminomethylquinazolin-4-yl)-(4-methoxyphenyl)methylamine, or a pharmaceutically acceptable salt thereof.

2. A pharmaceutical composition comprising a pharmaceutically acceptable carrier and an effective amount of the compound of claim 1.

3. The pharmaceutical composition of claim 2, further comprising at least one cancer chemotherapeutic agent other than the compound of claim 1, or a pharmaceutically acceptable salt of said at least one cancer chemotherapeutic agent.

4. The pharmaceutical composition of claim 3, wherein said at least one cancer chemotherapeutic agent is selected from alkylating agents, antimitotic agents, topoisomerase I inhibitors, topoisomerase II inhibitors, RNA/DNA antimetabolites, DNA antimetabolites, EGFR inhibitors, proteosome inhibitors, antibodies, and combinations thereof.

5. A composition effective to inhibit neoplasia comprising the compound of claim 1 in bioconjugation with at least one therapeutically useful antibody, growth factor, cytokine, or molecule that binds to a cell surface.

6. A method of treating or ameliorating neoplasm or cancer, said method comprising treating cells or a warm-blooded animal with an effective amount of the compound (2-aminomethylquinazolin-4-yl)-(4-methoxyphenyl)methylamine, or a pharmaceutically acceptable salt thereof.

7. The method of claim 6, further comprising administering at least one cancer chemotherapeutic agent other than said compound, or a pharmaceutically acceptable salt of said at least one cancer chemotherapeutic agent.

8. The method of claim 7, wherein said at least one cancer chemotherapeutic agent is selected from alkylating agents, antimitotic agents, topoisomerase I inhibitors, topoisomerase II inhibitors, RNA/DNA antimetabolites, DNA antimetabolites, EGFR inhibitors, proteosome inhibitors, antibodies, and combinations thereof.

9. The method of claim 7, wherein said at least one cancer chemotherapeutic agent comprises alkylating agents.

10. The method of claim 7, wherein said at least one cancer chemotherapeutic agent comprises antimitotic agents.

11. The method of claim 7, wherein said at least one cancer chemotherapeutic agent comprises topoisomerase I or II inhibitors.

12. The method of claim 7, wherein said at least one cancer chemotherapeutic agent comprises EGFR inhibitors.

13. The method of claim 7, wherein said at least one cancer chemotherapeutic agent comprise antimetabolites.

14. The method of claim 6, further comprising administering radiation therapy at the same time or at a different time as treating with said compound.

15. The method of claim 6, wherein treating said warm-blooded animal with cancer further comprises surgically removing said cancer and then administering an effective amount of said compound to said warm-blooded animal.

16. The method of claim 6, wherein said cancer comprises a drug-resistant cancer.

17. The method of claim 6, wherein said cancer comprises a primary cancer.

18. The method of claim 6, wherein said cancer comprises a metastatic cancer.

19. The method of claim 6, wherein said cancer is selected from Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

20. A method of preparing the compound (2-aminomethylquinazolin-4-yl)-(4-methoxyphenyl)methylamine, or a pharmaceutically acceptable salt thereof, said method comprising:
reacting 4-chloro-2-chloromethylquinazoline, or a salt thereof, with (4-methoxyphenyl)methylamine, or a salt thereof, under suitable conditions and with suitable reagents to form a first intermediate, (2-chloromethylquinazolin-4-yl)(4-methoxyphenyl)methylamine, or a salt thereof;
reacting said first intermediate with a phthalimide salt under suitable conditions and with suitable reagents to form a second intermediate, 2-{4-[(4-methoxyphenyl)methylamino]quinazolin-2-ylmethyl}isoindole-1,3-dione, or a salt thereof; and
reacting said second intermediate with an amine base under suitable conditions and with suitable reagents to form (2-aminomethylquinazolin-4-yl)-(4-methoxyphenyl)methylamine, or a pharmaceutically acceptable salt thereof.
